(19) United States
(12) Patent Application Publication
(10) Pub. No.: US 2015/0056674 A1
(43) Pub. Date: Feb. 26, 2015

(54) FERMENTATION PROCESS FOR CONVERGING CELLULOSIC SUGARS TO ETHANOL

(71) Applicant: CLEMSON UNIVERSITY, Clemson, SC (US)

(72) Inventors: Sarah W. Harcum, Clemson, SC (US); Yogender Gowtham, Clemson, SC (US)

(21) Appl. No.: 14/381,732

(22) PCT Filed: Feb. 21, 2013
(86) PCT No.: PCT/US2013/027006

§ 371 Date: Aug. 28, 2014

Related U.S. Application Data
(60) Provisional application No. 61/605,453, filed on Mar. 1, 2012.

(51) Int. Cl. C12P 7/14

(52) U.S. Cl. CPC C12P 7/14
USPC 435/162

(57) ABSTRACT

Methods using yeast, enzymes and bacteria to increase ethanol production rates from cellulosic sugars via multiple coupled fermentation processes.

### CROSS REFERENCE TO RELATED APPLICATION

The present application claims filing benefit of U.S. Provisional Patent Application Ser. No. 61/605,453 having a filing date of Mar. 1, 2012, which is incorporated herein by reference.

### STATEMENT REGARDING FEDERALLY SPONSORED RESEARCH

The U.S. Government has rights in this invention under Grant/Contract Number—Bioethanol Collaborative Award No. DE-FG36-08GO88071, Department of Energy, Golden, Colo.

### BACKGROUND

Biofuels are hydrocarbons derived from biological source material (biomass) that are useful in industrial applications, for instance directly as a fuel or as raw material for the production of chemicals. Biofuels, and particularly ethanol, provide an attractive alternative to current common fuels that are derived from non-renewable resources. Presently, most of the ethanol produced from biomass is derived from corn via the conversion of corn grain starch into glucose via enzymatic hydrolysis followed by fermentation to ethanol. Unfortunately, the increased demand for corn for biofuel production has led to higher prices for all products that utilize corn in production, which includes foods that span from meat and dairy products to processed foods that incorporate corn-based products such as high fructose corn syrup.

Prior ethanol extraction processes using fermentation are inefficient and do not maximize ethanol production. For instance, current cellulose to ethanol conversions cannot process sugars such as xylose efficiently, if at all. This results in waste of biomass as well as inefficient ethanol production. Further, some prior existing processes attempt to convert sugars such as xylose by incorporating recombinant organisms, such as recombinant yeast, that have been genetically modified to aid in ethanol production. However, engineering these organisms is a time and resource intensive process that typically requires numerous trial and error attempts to produce a useful organism. Further, such organisms are not as hardy as the naturally occurring species and have limited viability in the fermentation environment. Moreover, once produced, the impact of introducing the recombinant organism into the biosphere is unknown and far from predictable. This results in hesitancy to use these organisms as well as trepidation over what to do with waste materials that may contain viable organisms. Moreover, Federal approval may become an issue with using recombinant technologies. Additionally, some fermentation processes produce byproducts which may be used as feedstock for animals. However, the uncertainty associated with recombinant or modified organisms used in the fermentation process carries over to using these byproducts as feedstock, especially for animals that are later consumed by the populace.

Further, the cost of yeast used in fuel-grade fermentation processes may range from $180 to $220 per ton. This roughly increases costs by between $0.0645 to $0.075 per gallon of ethanol, assuming approximately 0.640/lb of yeast per gallon. The current international market for ethanol production is roughly 19.5 billion gallons per year and the base yeast value, at current manufacturer's prices, is estimated at $1.26 billion worldwide and $677 million in the U.S. Thus, yeast is a necessary, but costly, ingredient in the fermentation process for ethanol.

Previous processes for attempting to better utilize xylose sugars include U.S. Pat. No. 4,663,284 to Jeffries that discloses a process for producing ethanol from D-xylose by fermentation with xylose metabolizing yeasts, wherein small quantities of glucose are added to the fermentation medium during the fermentation process. Non-saccharomyces strain yeasts can ferment xylose if oxygen is allowed to be present in the fermentation; however, the ethanol production rate is very slow. The process of Jeffries further discloses that the addition of glucose to these oxygen mediated fermentations improves the yield of the fermentation; however, the process is not enzyme mediated.

U.S. Pat. No. 4,511,656 to Gong pertains to a method for producing ethanol directly from D-xylose through fermentation of D-xylose by yeast mutants. The process further provides for directly and simultaneously obtaining ethanol from a mixture of cellulose and hemicellulose through yeast fermentation of D-glucose and D-xylose; however, no enzymes are used.

In U.S. Pat. No. 4,490,468 to Gong et al., there is described an anaerobic fermentation of xylulose previously obtained by isomerization of xylose, and example 6 thereof briefly mentions the possibility of simultaneous isomerization and fermentation of xylose; however, the process is not combined in any way with the fermentation of cellulosic sugars or the conversion of xylose via a bacterial fermentation.

U.S. Pat. No. 4,368,268 to Gong relates to a process for the production of ethanol from xylulose. The process includes isomerizing the xylose to xylulose and fermenting the xylulose to ethanol. Essentially, this process is the fermentation of xylose or xylose and other sugars in hemicellulose hydrolysates by mutant strains of yeast, either aerobically or anaerobically; however, hemicellulose does not refer to cellulose but to extracts obtained by pretreatment of materials that contain cellulose. The sugars obtained are soluble sugars (in most cases mostly xylose). Cellulose is not soluble and must be enzymatically digested to produce soluble sugars. The fermentations in this patent are not anaerobic but oxygen mediated rather than enzyme mediated fermentations.

U.S. Pat. No. 4,840,903 to Wu discloses a process for the production of ethanol by a fungal strain capable of slowly degrading and fermenting cellulose, xylose, and a number of other sugars. Like simultaneous saccharification and fermentation (SSF) of cellulose, cellulase enzymes were added to the fermentations to produce glucose from cellulose; however, the fermentations are not a combination of enzymatic isomerization of xylose to xylulose, a fungal strain rather than ethanol tolerant yeast was used for the fermentation, and fungal strains take much longer to grow and ferment, and these longer lengths of time or slow rates are unacceptable for industrial purposes.

U.S. Pat. No. 5,372,939 to Lastick discloses producing ethanol from a mixed stream of xylose and cellulose using enzymes to convert these carbohydrates to fermentable sugars under predetermined conditions. This is done by the simultaneous conversion of cellulose to glucose, using cellulase enzymes, and the conversion of xylose to xylulose, using the enzyme xylose isomerase in the presence of Schizosaccharomyces pombe ATCC No. 2476. The enzymatic processes allow for these fermentable sugars, glucose and xylulose, to be converted by yeast to ethanol in the same fermentation. The continuous conversion of the sugars to ethanol by the fermenting yeast is key to the process because the activities of the enzymes are inhibited in the presence of their products, glucose (and the disaccharide cellobiose) and xylulose. The intent is to produce ethanol from xylose using cellulosic biomass waste without having to ferment separately a stream containing soluble xylose in an enzyme mediated process.

What is needed in the art are environmentally friendly, low cost methods for production of ethanol with improved production and efficiency over past processes that enables capture of sugars that are currently not captured efficiently as well as the ability to lower the costs associated with the fermentation process.

### SUMMARY

A multiple stage fermentation process is disclosed. The process can include contacting a mixture comprising multiple sugars with one or more strains of yeast, a first portion of the sugars being converted by the yeast according to a first fermentation process. The process can also include contacting a second portion of the sugars with one or more strains of bacteria, the second portion of the sugars being converted by the bacteria according to a second fermentation process. The process also includes contacting at least one of the first portion of the sugars and the second portion of the sugars with an enzyme, wherein one or more of the sugars is a substrate for the enzyme.

### BRIEF DESCRIPTION OF THE DRAWINGS

A full and enabling disclosure, including the best mode thereof, to one of ordinary skill in the art, is set forth more particularly in the remainder of the specification, including reference to the accompanying Figures, in which:

FIG. 1 is a schematic diagram of one embodiment of a multiple fermentation process of the disclosure.

FIG. 2 shows comparison charts displaying varying ethanol production and glucose consumption values.

FIG. 3A is a graphic illustration of growth density of the yeast Pichia stipitis on different media.

FIG. 3B is a graphic illustration of growth density of the bacterium E. coli on a media lacking essential micronutrients required by yeast species.

FIG. 4 illustrates a comparison between Saccharomyces pastorianus and Saccharomyces cerevisiae with respect to ethanol concentration on different media.

FIG. 5 illustrates growth, glucose, and ethanol concentration profiles for Saccharomyces pastorianus and Saccharomyces cerevisiae cultured in minimal medium with varying xylose and xylose isomerase levels.

FIG. 6 presents glucose consumption profiles for S. pastorianus on corn stover hydrolysate and synthetic biomass sugars in the presence of heat-killed E. coli extract, xylose isomerase, and minimal medium.

FIG. 7 presents xylose profiles for S. pastorianus on corn stover hydrolysate and synthetic biomass sugars in the presence of E. coli extract, xylose isomerase, and minimal medium.

FIG. 8 presents ethanol profiles for S. pastorianus on corn stover hydrolysate and synthetic biomass sugars in the presence of E. coli extract, xylose isomerase, and minimal medium.

FIG. 9 presents glucose, xylose, and ethanol concentration profiles for S. pastorianus in the presence of heat-killed E. coli extract from a fed-batch fermentation on synthetic sugar biomass sugars. Additionally, the glucose consumed and ethanol produced profiles are shown.

### DETAILED DESCRIPTION

Reference will now be made in detail to various embodiments of the presently disclosed subject matter, one or more examples of which are set forth below. Each embodiment is provided by way of explanation, not limitation, of the subject matter. In fact, it will be apparent to those skilled in the art that various modifications and variations may be made to the present disclosure without departing from the scope or spirit of the disclosure. For instance, features illustrated or described as part of one embodiment, may be used in another embodiment to yield a still further embodiment. Thus, it is intended that the present disclosure cover such modifications and variations as come within the scope of the appended claims and their equivalents.

In general, the present disclosure is directed to a multiple stage fermentation process, including both yeast and bacteria mediated fermentation processes carried out in conjunction with one or more useful enzymes, to consume sugars, and/or cellulosic-derived sugars in order to produce products such as alcohols and organic byproducts.

The fermentation process may include, without limitation, fermentation methods or processes used to produce any fermentation product, including alcohols (e.g., ethanol, methanol, butanol); organic acids (e.g., citric acid, acetic acid, itaconic acid, lactic acid, gluconic acid); ketones (e.g., acetone); amino acids (e.g., glutamic acid); gases (e.g., H₂ and CO₂); antibiotics (e.g., penicillin and tetracycline); enzymes; vitamins (e.g., riboflavin, B₁₂, beta-carotene); and hormones. The fermentation process may also be a fermentation step used in the consumable alcohol industry (e.g., beer and wine), dairy industry (e.g., fermented dairy products), leather industry and tobacco industry. In one embodiment, the fermentation process is an alcohol fermentation process. In one embodiment of the present disclosure, the process for yeast fermentation can be both aerobic and anaerobic, as there is oxygen in the beginning media due to naturally occurring oxygen. However, oxygen need not be added during the main fermentation. With respect to the bacterial fermentation stage, oxygen may or may not be supplied. In one embodiment, oxygen is supplied in the bacterial fermentation stage. In a further embodiment, the process may originally have oxygen supplied to the bacteria but then the bacterial fermentation stage can become anaerobic as oxygen is consumed faster than it is supplied.

In one embodiment, sugars that may be consumed by the multiple stage fermentation process may have between three and seven carbon atoms in their structure, for instance five or six carbon atoms in their structure. For purposes of example only and not intended to be limiting, the sugars may include but are not limited to glucose, fructose, mannose, galactose, xylose, xylulose, and arabinose as well as mixtures of sugars.

The sugars may be obtained from any feedstock source including, without limitation, sources such as corn stover, switchgrass, miscanthus, woodchips and the byproducts of lawn and tree maintenance. For instance, cellulosic biomass such as switchgrass (Panicum virgatum L.) is an alternative feedstock for ethanol production and can be utilized in the multiple stage fermentation process. Cellulosic feedstock hydrolysates contain high levels of xylose, which needs to be converted to ethanol to meet economic feasibility. Due to the high cellulose content and rapid growth of switchgrass, the US Department of Energy has identified it as a model herbaceous renewable energy crop. In order for switchgrass biomass to be fermented to ethanol, it first needs to be treated to release the sugars in a two-step process termed pre-treatment and hydrolysis. Pretreated hydrolyzed switchgrass contains approximately 60% glucose and 40% xylose. The ability of organisms to completely ferment these mixed sugars to ethanol is an important aspect of economic feasibility for the disclosed multiple stage fermentation process.

The feedstock source for the sugars may also comprise lignocellulose-containing material that primarily consists of cellulose, hemicellulose, and lignin and is often referred to as “biomass”. The structure of lignocellulose is not directly accessible to enzymatic hydrolysis. Therefore, the lignocellulose may be pre-treated, e.g., by acid hydrolysis under adequate conditions of pressure and temperature, in order to break the lignin seal and disrupt the crystalline structure of cellulose. This causes solubilization and saccharification of the hemicellulose fraction. The cellulose fraction can then be hydrolyzed enzymatically, e.g., by cellulase enzymes, to convert the carbohydrate polymers into fermentable sugars, which may be fermented into a desired fermentation product, such as ethanol, which may optionally be recovered, e.g., by distillation.

A lignocellulose-containing feedstock material may be any material containing lignocellulose. In one embodiment the lignocellulose-containing material contains 30-90 weight percent, at least 50 weight percent, at least 70 weight percent, or at least 90 weight percent, lignocellulose. It is to be understood that the lignocellulose-containing material may also comprise other constituents such as cellulosic material, including cellulose and hemicellulose, and may also comprise constituents such as proteinaceous material, starch, and sugars such as fermentable sugars and/or un-fermentable sugars.

Lignocellulose-containing material is generally found in the stems, leaves, hulls, husks, and cobs of plants or in the leaves, branches, and wood of trees. Lignocellulose-containing material can also be, but is not limited to, herbaceous material, agricultural residues, forestry residues, municipal solid wastes, waste paper, and pulp and paper mill residues. It is to be understood that lignocellulose-containing material may be in the form of plant cell wall material containing lignin, cellulose, and hemi-cellulose in a mixed matrix.

Suitable yeasts for use in the multiple stage fermentation process may include species within the genus Saccharaomyces, including for purposes of example only and not intended to be limiting, Saccharomyces boulardii. Saccharomyces bayanus, Saccharomyces boulardii, Saccharomyces bulderi, Saccharomyces cariocanus, Saccharomyces cariocus, Saccharomyces cerevisiae, Saccharomyces chevalieri, Saccharomyces dairenensis, Saccharomyces ellipsoideus, Saccharomyces eubayanus, Saccharomyces exiguus, Saccharomyces florentinus, Saccharomyces kluyveri, Saccharomyces martiniae, Saccharomyces monacensis, Saccharomyces norbensis, Saccharomyces paradoxus, Saccharomyces pastorianus, Saccharomyces spencerorum, Saccharomyces turicensis, Saccharomyces unisporus, Saccharomyces uvarum, Saccharomyces zonatus. These yeasts may be used alone or in combination with one another or other compatible yeasts. Two exemplary yeast species that may be used together are Saccharomyces cerevisiae and Saccharomyces pastoranius.

Saccharomyces cerevisiae is the most commonly used species for biofuel production. However, there are several other Saccharomyces species with robust growth rates and high ethanol tolerance. Saccharomyces pastorianus is currently used in the lager brewing industry and is responsible for roughly 90 percent of the beer market.

Saccharomyces pastorianus is a hybrid of the traditional brewing yeasts Saccharomyces bayanus and Saccharomyces cerevisiae. Saccharomyces cerevisiae and Saccharomyces bayanus often share environmental niches and are found together in wine and beer fermentations. Generally, Saccharomyces cerevisiae grows and produces ethanol between 30° C. and 35° C., while Saccharomyces bayanus can grow and produce ethanol at temperatures ranging from 1° C. to 30° C. The Saccharomyces pastorianus strain has retained the ability to ferment sugars at low temperatures (8° C. to 15° C.) from Saccharomyces bayanus. Additionally, Saccharomyces pastorianus can tolerate the high gravity conditions of brewing that include high ethanol and sugar concentrations and can ferment sugars at temperatures up to 34° C. Saccharomyces pastorianus has separate glucose and fructose transporters, whereas the single Saccharomyces cerevisiae hexose transporter significantly prefers glucose to fructose. Saccharomyces pastorianus has sufficient favorable growth and ethanol tolerance characteristics to be considered a fermentative candidate species for cellulosic ethanol production, and may provide increased tolerance to biomass sugar hydrolysate inhibitors compared to Saccharomyces cerevisiae.

Other suitable yeasts may include species within the genus Candida, including for purposes of example only and not intended to be limiting, Candida albicans, Candida ascalaphidarum, Candida amphixiae, Candida Antarctica, Candida argentea, Candida atlantica, Candida atmosphaerica, Candida blattae, Candida carpophila, Candida carvajalis, Candida cerambycidarum, Candida chauliodes, Candida corydalis, Candida dosseyi, Candida dubliniensis, Candida ergatensis, Candida fructus, Candida glabrata, Candida fermentati, Candida guilliermondii, Candida haemulonii, Candida insectamens, Candida insectorum, Candida intermedia, Candida jeffresii, Candida kefyr, Candida krusei, Candida lusitaniae, Candida lyxosophila, Candida maltose, Candida marina, Candida membranifaciens, Candida milleri, Candida oleophila, Candida oregonensis, Candida parapsilosis, Candida quercitrusa, Candida rugosa, Candida sake, Candida shehatea, Candida temnochilae, Candida tenuis, Candida theae, Candida tropicalis, Candida tsuchiyae, Candida sinolaborantium, Candida sojae, Candida subhashii, Candida viswanathii, and Candida utilis.

The process is directed to consumption of the primary substrate sugars of a feedstock, such as for purposes of example only, glucose in conjunction with xylose, as in the case of a lignocellulose-containing feedstock, and production of ethanol from same. The process includes a yeast fermentation process that may utilize, e.g., yeast of the Saccharomyces genus. However, for the process to be economical, it is considered essential that most or all of the feedstock sugars, e.g., xylose from cellulosic biomass, be converted.

To improve conversion of the feedstock, one or more enzymes can be introduced to the process, for instance in conjunction with the yeast fermentation step. For example, all yeasts of the genus Saccharomyces lack the gene that produces the enzyme xylose isomerase. However, conversion of xylose to xylulose is necessary for improved economic feasibility of the use of certain feedstock and in particular, lignocellulose feedstock. Accordingly, enzymes for which one or more sugars is a substrate can be incorporated into a process. Utilization of such enzymes can improve conversion of feedstock sugars that are not highly converted by the yeast and/or bacteria of the process. By way of example, when considering a feedstock that includes a large proportion of xylose, a xylose isomerase enzyme can be incorporated into the process, for instance in conjunction with the yeast fermentation step. In one embodiment, the enzyme(s) can be immobilized, for instance through formation of a granular immobilized enzyme as described in U.S. Pat. No. 4,687,742 to Skoet et al., which is incorporated herein by reference.

Xylose isomerases (D-xylose ketoisomerase) (E.G. 5.3.1.5.) are enzymes that catalyze the reversible isomerization reaction of D-xylose to D-xylulose. Some xylose isomerases also convert the reversible isomerization of D-glucose to D-fructose. Therefore, xylose isomerase is sometimes referred to as “glucose isomerase.” A xylose isomerase used in a process of the present disclosure may be any enzyme having xylose isomerase activity and may be derived from any sources, preferably bacterial or fungal origin, such as filamentous fungi or yeast. Examples of bacterial xylose isomerases include the ones belonging to the genera Streptomyces, Actinoplanes, Bacillus and Flavobacterium, and Thermotoga, including T. neapolitana (Vieille et al. Appl. Environ. Microbiol. 1995, 61 (5), 1867-1875) and T. maritima. Examples of fungal xylose isomerases are derived species of Basidiomycetes.

In one embodiment, xylose isomerase may be derived from a strain of yeast genus Candida, preferably a strain of Candida boidinii, especially the Candida boidinii xylose isomerase disclosed by, e.g., Vongsuvanlert et al., (1988), Agric. Biol. Chem., 52(7): 1817-1824. The xylose isomerase may preferably be derived from a strain of Candida boidinii (Kloeckera 2201), deposited as DSM 70034 and ATCC 48180, disclosed in Ogata et al. Agric. Biol. Chem., Vol. 33, p. 1519-1520 or Vongsuvanlert et al. (1988) Agric. Biol. Chem., 52(2), p. 1519-1520.

In another embodiment the xylose isomerase is derived from a strain of Streptomyces, e.g., derived from a strain of Streptomyces murinus (U.S. Pat. No. 4,687,742); S. flavovirens, S. albus, S. achromogenus, S. echinatus, S. wedmorensis all disclosed in U.S. Pat. No. 3,616,221. Other xylose isomerases are disclosed in U.S. Pat. No. 3,622,463, U.S. Pat. No. 4,351,903, U.S. Pat. No. 4,137,126, U.S. Pat. No. 3,625,828, and all of the above patents are hereby incorporated herein by reference. The xylose isomerase may be either in immobilized or liquid form. In a preferred embodiment, the xylose isomerase is immobilized. Xylose isomerase may be added to provide an activity level in the range from 0.01-200 units per liter fermentation media. Examples of commercially available xylose isomerases include SWEETZYME™ T from Novozymes A/S, Denmark (greater than 350 units per gram enzyme). One unit is the amount of enzyme, which converts glucose to fructose at an initial rate of 1 micromole per minute at standard analytical conditions. The standard conditions include Glucose concentration: 45% w/w, pH: 7.5, Temperature: 60 degrees Celsius, Mg²⁺ concentration: 99 mg/L (1.0 g/L MgSO₄.7H₂O), Ca²⁺ concentration <2 ppm, Activator, SO₂ concentration: 100 ppm (0.18 g/L NaS₂O₅), Buffer, Na₂CO₃, concentration: 2 mM.

The enzymes for use in the process are not limited to xylose isomerases. Enzymes such as hemicellulase, cellulase or combinations of these and other enzymes may be used. Other enzymes that may be used include Alpha-Amylases, Bacterial Alpha-Amylases, Bacterial Hybrid Alpha-Amylases, Fungal Alpha-Amylases, Fungal Hybrid Alpha-Amylases, Carbohydrate-Source Generating Enzymes including Glucoamylases, Beta-Amylases, Maltogenic Amylases, and Proteases. Any hemicellulase suitable for use in hydrolyzing hemicellulose into xylose may be used. Exemplary hemicellulases for use can include xylanases, arabinofuranosidases, acetyl xylan esterase, feruloyl esterase, glucuronidases, endo-galactanase, mannases, endo or exo arabinases, exo-galactanses, and mixtures thereof. In one embodiment, the hemicellulase for use in the present invention is an exo-acting hemicellulose. For instance, the hemicellulase can be an exo-acting hemicellulase that has the ability to hydrolyze hemicellulose under acidic conditions of below pH 7, for example pH 3-7. An example of hemicellulase includes VISCOZYME™ (available from Novozymes NS, Denmark). The hemicellulase can be added in an amount effective to hydrolyze hemicellulose into xylose, such as, in amounts from about 0.001 to 0.5 wt. % of total solids (TS), more preferably from about 0.05 to 0.5 wt. % of total solids.

Any cellulase that is capable of hydrolyzing cellulose into glucose may be used according to the present invention. The cellulase activity used according to the invention may be derived from any suitable origin; preferably, the cellulase is of microbial origin, such as derivable from a strain of a filamentous fungus (e.g., Aspergillus, Trichoderma, Humicola, Fusarium, Thielavia). Preferably, the cellulase composition acts on both cellulosic and lignocellulosic material. Preferred cellulases for use in the present invention include exo-acting cellulases and cellobiases, and combinations thereof. More preferably, the treatment involves the combination of an exo-acting cellulase and a cellobiase. Preferably, the cellulases have the ability to hydrolyze cellulose or lignocellulose under acidic conditions of below pH 7. Examples of cellulases suitable for use in the present invention include, for example, CELLULCLAST™ (available from Novozymes NS), NOVOZYM™ 188 (available from Novozymes A/S). Other commercially available preparations comprising cellulose, which may be used include CELLUZYME™, CEREFLO™ and ULTRAFLO™ (Novozymes NS), LAMINEX™ and SPEZYME™ CP (Genencor Int.) and ROHAMENT™ 7069 W (from Rohm GmbH).

Following the yeast fermentation process, the yeast may be separated from the other materials and fermentation products (e.g., ethanol) may be removed from the stream. The sugars remaining in the yeast fermentation product stream following these separations may then be supplied to a bacterial fermentation stage. The two fermentation processes need not take place in separate steps and vessels, however. For example, in one embodiment, the yeast fermentation and the bacterial fermentation can take place simultaneously in a single vessel, and the separation processes can be carried out following the two stages of fermentation. Moreover, the separation processes can take place at any point in the process such as following separate yeast and bacterial fermentation processes, following a single fermentation process that includes the combined yeast and bacterial fermentations in a single vessel, or following one or more stages of a multiple stage fermentation process that includes more than one yeast mediated fermentation and/or more than one bacteria mediated fermentation.

In the bacterial fermentation stage, the unconverted sugars are introduced to bacteria. As previously mentioned, the action of enzymes can be incorporated at any point in the process. Thus, in one embodiment, sugars remaining after an initial yeast fermentation process including the yeast mediated fermentation carried out in the presence of one or more enzymes are introduced to a bacterial fermentation process, and this mixture may be allowed to ferment via action of the bacteria. In another embodiment, the initial yeast fermentation process can be carried out without the inclusion of added enzymes, and the sugars remaining after the initial yeast fermentation can include sugars that are unconverted by the yeast. In this embodiment, one or more useful enzymes may be added to the bacterial fermentation process. Of course, the same or different enzymes may also be added to both the yeast fermentation and the bacterial fermentation. In yet another embodiment, all of the yeast, bacteria, and enzymes can be contacted with the feedstock in a single vessel, and the multiple stage fermentation can take place simultaneously in a single-step process. The bacterial fermentation step may be allowed to progress for a suitable period of time, including but not limited to 5, 10, 15, 20, 25, 30, or 35 hours, or values between these time intervals. One exemplary bacterial fermentation period is 24 hours.

Suitable bacteria for a bacterial fermentation stage may include bacteria species from the genus Escherichia, genus Salmonella, genus Bacillus, and genus Zymomonas. In one embodiment, bacteria from the genus Escherichia can be utilized. Suitable bacteria from the Escherichia genus include bacteria species such as Escherichia albertii, Escherichia aurescens, Escherichia blattae, Escherichia coli, Escherichia coli (E. Coli), Escherichia coli communior, Escherichia colimigula, Escherichia coli-mutabile, Escherichia colii, Escherichia coli K-12, Escherichia communior, Escherichia ellingeri, Escherichia fergusonii, Escherichia freundii, Escherichia hermanii, Escherichia intermedia, Escherichia intermedium, Escherichia metacoli, Escherichia noctuarii, Escherichia sphingidis, and Escherichia vulneris. These bacteria may be used alone or in combination with one another. One preferable bacteria species is Escherichia coli K-12.

Once the bacterial fermentation stage is complete, the bacteria used in the bacterial fermentation process may be neutralized or deactivated to yield a killed bacterial slurry. Possible methods for killing the bacteria include heat treating, mechanical destruction, chemical application, antibiotic application, etc., as known to those of skill of the art.

After the bacteria are killed, the remaining bacterial slurry can then be introduced to a second yeast fermentation process. This may be the same yeast fermentation process as initially employed, and may occur in the same vessel or a different vessel. The yeast employed in this fermentation process may be the same yeast or combination of yeasts used in the initial yeast fermentation stage or may be a different combination of yeasts.

The media used to carry the feedstock and mixtures through the system can vary depending upon the specific parameters of the system and process. For instance a rich media, a minimal media, or a combination thereof may be used. Rich media is the name used for typical yeast media that may be adapted from the YPD medium of Gong et al. (1981). It typically contains 10 g/L yeast extract, 20 g/L peptone, and 50 g/L glucose. This would constitute 100% Rich Media. Rich Media may be used to provide micronutrients not in minimal media that are needed for yeast growth. Minimal medium generally includes a phosphate buffer (KH₂PO₄, and (NH₄)₂HPO₄), citric acid, magnesium sulfate, trace metals (MnCl₂.4H₂O, Zn (CH₃COO)₂.2H₂O, H₃BO₃, Na₂MoO₄.2 H₂O, CoCl₂.6H₂O, CuCl₂.2H₂O, and EDTA), and iron (III) citrate at pH 5.8. A combination of media can be used, for instance a combination of rich media and minimal media in a weight ratio of from about 1:99 to 99:1.

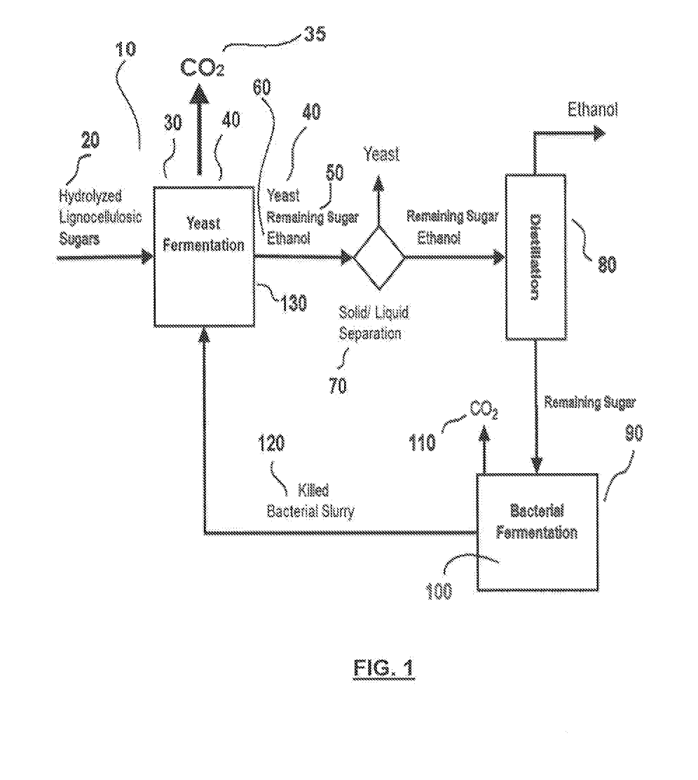

FIG. 1 illustrates one embodiment of a multiple stage fermentation process in a schematic view. As FIG. 1 shows, a multiple fermentation process 10 may begin with hydrolyzed lignocellulosic sugar feedstock 20 being introduced to a fermentation vessel 30 containing yeast 40. The fermentation vessel can include one or more yeast species and optionally one or more added enzymes, as discussed above. Carbon dioxide (CO₂) 35 is produced by the fermentation and can be captured. After yeast fermentation occurs, the product stream including yeast 40, remaining sugar 50 and ethanol fermentation products 60 are removed from the fermentation vessel 30 and undergo solid/liquid separation 70 to recapture the yeast. This may be accomplished by any means known to those of skill in the art. The recaptured yeast can be reused for additional yeast fermentation processes.

After solid/liquid separation 70, the remaining mixture including unconverted or partially converted sugar 50 and ethanol 60 may undergo separation such as via distillation 80 to separate the ethanol 60 from the remaining sugar 50. The remaining sugar 50 may then be introduced to a bacterial fermentation chamber 90 containing bacteria 100 and optionally one or more useful enzymes. Bacterial fermentation is then allowed to occur. CO₂ 110 may be vented from the bacterial fermentation chamber 90 and captured. Subsequent to bacterial fermentation, bacteria 100 are deactivated or killed to produce a killed bacterial slurry 120. The killed bacterial slurry 120 may then be introduced to a yeast fermentation vessel, such as vessel 30 or a different vessel (not shown).

The killed bacterial slurry 120 then undergoes an additional yeast fermentation. This yeast fermentation may include one or more yeast strains and one or more enzymes, e.g., xylose isomerase, employed in the initial yeast fermentation or may utilize different yeast(s), or combination of yeasts as well as different enzymes or combination of enzymes. The resulting fermented killed bacterial slurry 130 may then undergo solid/liquid separation 70 and/or distillation 80 to separate yeast and ethanol produced from the second yeast fermentation from any remaining solid product. While FIG. 1 describes a recycle system with a single yeast fermentation and a single bacterial fermentation in each cycle, the process is not so limited and may include additional yeast and bacterial fermentation stages, either yeast or bacterial, as well as variations on the order of the various fermentation stages in each cycle of a process.

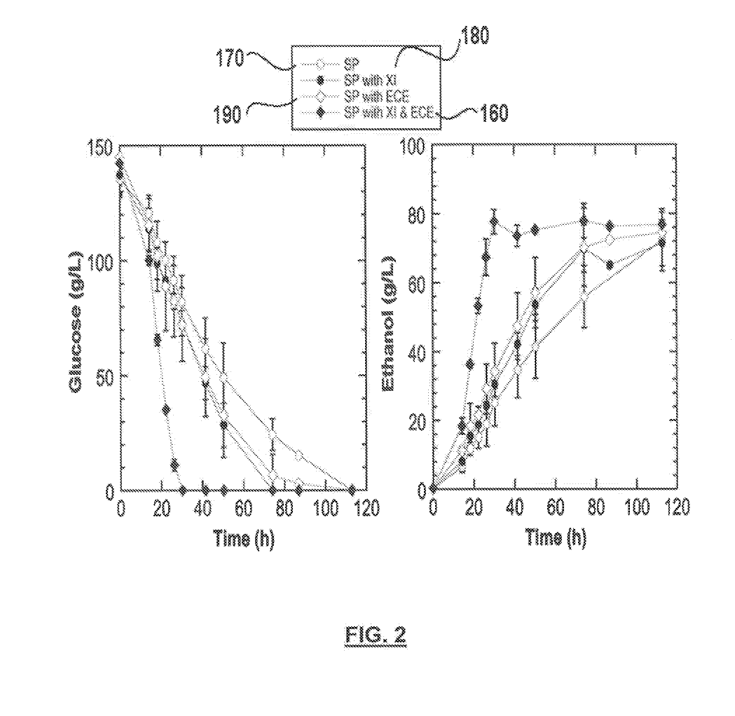

FIG. 2 shows a comparison chart displaying varying ethanol production and glucose consumption values for a system as described herein. FIG. 2 illustrates that by introducing the yeast Saccharomyces pastoranius to xylose isomerase in the presence of killed Escherichia coli slurry 160, both glucose consumption and ethanol production are improved as compared to Saccharomyces pastoranius acting alone 170, or Saccharomyces pastoranius acting with only xylose isomerase 180 or Saccharomyces pastoranius acting with only killed Escherichia coli slurry 190. Further, as illustrated by FIG. 2, both glucose consumption and ethanol production are markedly increased when the Saccharomyces pastoranius, xylose isomerase and Escherichia coli combination 160 is used. Meanwhile, FIG. 2 shows that the time required for ethanol production is lower for combination 160.

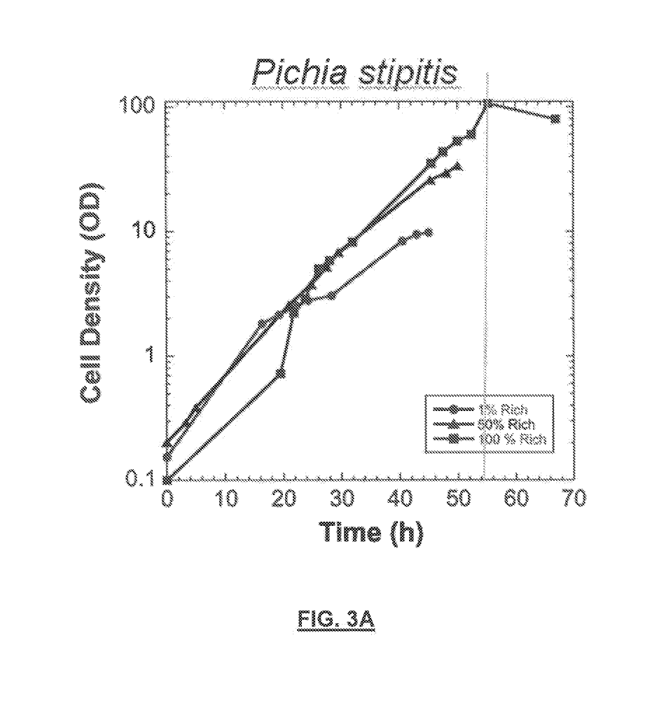

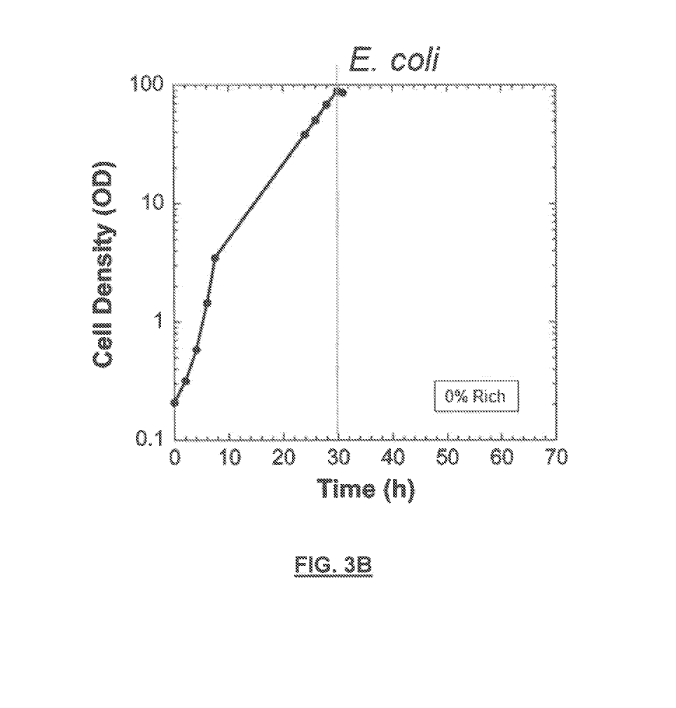

FIGS. 3A and 3B, meanwhile, illustrate that Pichia stipitis, (aka Scheffersomyces stipitis) a species of yeast and a distant relative to brewer's yeast, Saccharomyces cerevisiae, grows much slower on various Rich media at 1%, 50% and 100% concentrations than Escherichia coli on a 0% Rich media. Thereby showing the ease of producing Escherichia coli stock for use in a bacterial fermentation stage and also improving production times and lowering costs as the media is not required to be rich media as opposed to cultivating Pichia stipitis on same.

Table 1, shown below, compares the ethanol production that may be accomplished when using xylose isomerase in association with Saccharomyces pastoranius as well as Saccharomyces cerevisiae alone or in combination with xylose isomerase and the recycle system as described herein. These comparisons demonstrate that xylose isomerase may increase ethanol productivity for both yeast species by increasing the glucose consumption rate. Further, the fast ethanol production rates observed by Yuan et al. (2011) are due to high nutrient loading, which when fully accounted in the overall yields, significantly decreases the ethanol yields from the total nutrients utilized.

TABLE 1

| | Study | | | | | | |
|---|---|---|---|---|---|---|---|
| | Chiang et al., | Gong et al., | | | Yuan et al., | Traditional | Recycle |
| | 1981 | 1981 | | | 2011 | Fermentation | Fermentation |
| | Organism | | | | | | |
| | S. cerevisiae | S. cerevisiae | | | S. cerevisiae | S. pastorianus | S. pastorianus |
| Xylose Isomerase | Xylulose pre- | NA | Xylulose pre- | Immobilized | Bilayer | Immobilized | Immobilized |
| | converted | | converted | | Immobilized | | |
| Xylose Isomerase Used (g/L) | NA | NA | NA | 100 | 18 | 5 | 5 |
| Inputs | | | | | | | |
| Total Nutrients (g/L)* | 210 | 67 | 41 | 151 | 589 | 200 | 225 |
| Glucose (g/L) | | 46 | | | 130 | 160 | 150 |
| Xylose (g/L) | | | | 120 | 50 | 40 | 75 |
| Xylulose (g/L) | 60 | | 20 | | | | |
| Rich components (g/L) | | 11 | 11 | 11 | 9 | 0.3 | 0.3 |
| Biomass (g/L) | 75 | 5 | 5 | 10 | 200 | 0.025 | 0.025 |
| Outputs | | | | | | | |
| Ethanol (g/L) | 12.5 | 21 | 12 | 18 | 70 | 70 | 78 |
| Fermentation Time (h) | 6 | 12 | 45 | 10 | 30 | 65 | 30 |
| Biomass (g/L) | NR | NR | NR | NR | NR | 6 | NR |
| Overall | | | | | | | |
| Ethanol production rate (g/L*h) | 2.08 | 1.75 | 0.27 | 1.80 | 2.33 | 1.08 | 26 |
| Yield from Total Nutrients (g/g) | 0.06 | 0.31 | 0.29 | 0.12 | 0.12 | 0.35 | 0.35 |
| Yield excluding Biomass (g/g)** | 0.21 | 0.37 | 0.39 | 0.14 | 0.37 | 0.35 | 0.35 |

*Total Nutrients is the sum of the sugars and rich components added to the fermentaion plus 2 times the biomass inoculum.
**(Nutrient Input + Biomass Input) equals the sum of the sugars and rich made components.
NR—Not Reported
NA—Not Applicable

Table 2 illustrates a comparison of ethanol yield from previously known fermentation processes and the recycle multiple stage fermentation process as described herein. As can be see, the various processes utilized Saccharomyces pastoranius with exogenous xylose isomerase as well as recombinant Saccharomyces cerevisiae.

TABLE 2

| | Study | | | | | |
|---|---|---|---|---|---|---|
| | Almedia et al., | Bera et al., | Guo et al., | Ha et al., | Traditional | Recycle |
| | 2009 | 2011 | 2011 | 2011 | Fermentation | Fermentation |
| | Organism | | | | | |
| | Recombinant | Recombinant | Recombinant | Recombinant | | |
| | S. cerevisiae | S. cerevisiae | S. cerevisiae | S. cerevisiae | S. pastorianus | S. pastorianus |
| Xylose Isomerase | NA | NA | NA | NA | Immobilized | Immobilized |
| Xylose Isomerase Used (g/L) | NA | NA | NA | NA | 5 | 5 |
| Inputs | | | | | | |
| Total Nutrients (g/L)* | 70 | 110 | 81 | 200 | 200 | 225 |
| Glucose (g/L) | 20 | | | | 160 | 150 |
| Xylose (g/L) | 50 | 70 | 50 | 60 | 40 | 75 |
| Cellobiose (g/L) | | | | 100 | | |
| Rich components (g/L) | | 30 | 30 | 30 | 0.3 | 0.3 |
| Biomass (g/L) | 0.1 | 4.76 | 0.25 | 5 | 0.025 | 0.025 |
| Outputs | | | | | | |
| Ethanol (g/L) | 20 | 27 | 25 | 60 | 70 | 78 |
| Fermentation Time (h) | 90 | 25 | 28 | 72 | 65 | 30 |
| Biomass (g/L) | 3 | NR | 3.5 | 17 | 6 | NR |
| Overall | | | | | | |
| Ethanol production rate (g/L*h) | 0.22 | 1.08 | 0.89 | 0.83 | 1.08 | 2.6 |
| Yield from Total nutrients (g/g) | 0.28 | 0.25 | 0.31 | 0.30 | 0.35 | 0.35 |
| Yield excluding Biomass (g/g)** | 0.29 | 0.27 | 0.31 | 0.32 | 0.35 | 0.35 |

*Total Nutrients is the sum of the sugars and rich components added to the fermentation plus 2 times the biomass inoculum.
**(Nutrient inputs + Biomass Input)equals the sum of the sugars and rich made components
NR—Not Reported
NA—Not Applicable

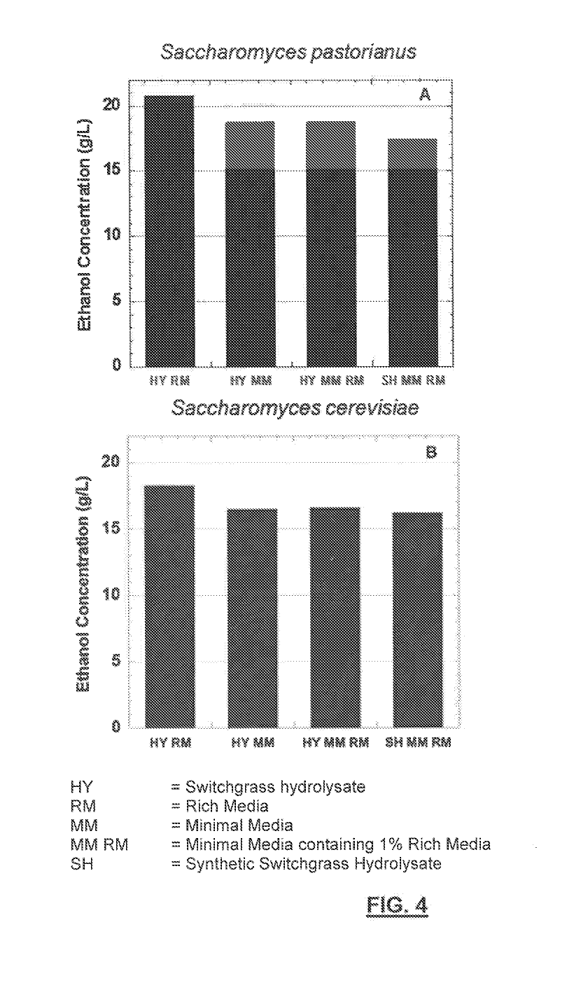

FIG. 4 illustrates results of the disclosed process using different feedstock (either switchgrass hydrolysate or synthetic switchgrass hydrolysate) and utilizing different media (minimal media, rich media, or a combination thereof containing 1% rich media) for two different yeast strains in the yeast fermentation step: Saccharomyces pastorianus and Saccharomyces cerevisiae. As can be seen, there was greater ethanol yield from Saccharomyces pastorianus.

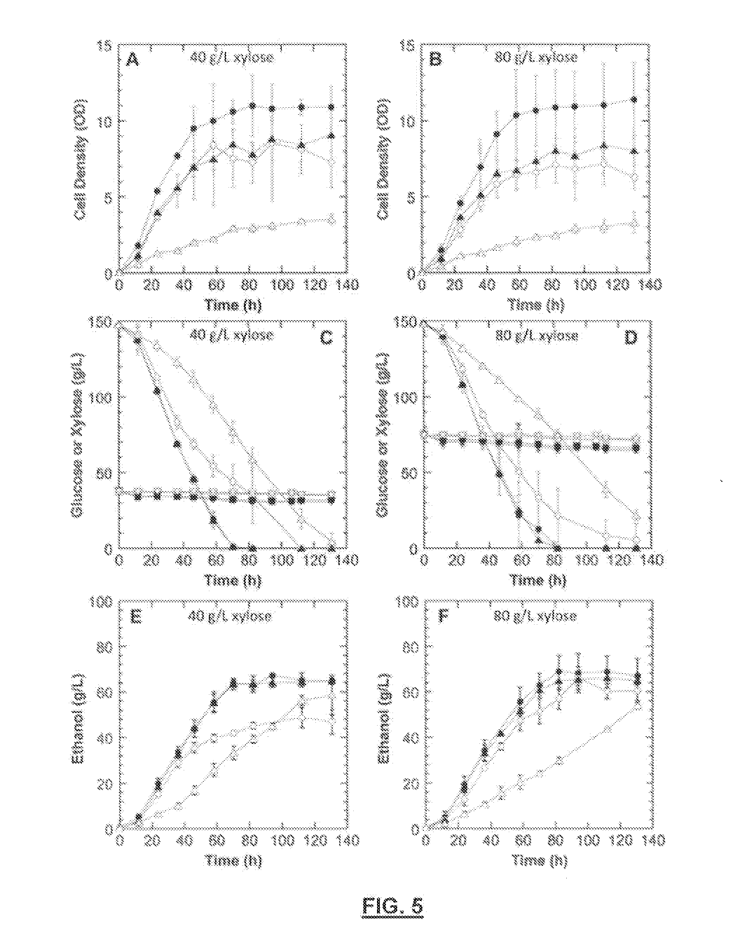

FIG. 5 illustrates growth, glucose, and ethanol concentration profiles for Saccharomyces pastorianus and Saccharomyces cerevisiae cultured in minimal medium with 160 g/L glucose and varying xylose and xylose isomerase levels. The growth profiles are shown in panels A and B. The glucose (•,◯,▴,Δ) and xylose (▪,□,♦,⋄) profiles are shown in panels C and D. The ethanol concentration profiles are shown in panels E and F. The medium contained 40 g/L xylose (A, C, E) or 80 g/L xylose (B, C, F). Saccharomyces pastorianus (•,◯,▪,□) and Saccharomyces cerevisiae (▴,Δ,♦,⋄) are shown with 5 g/L xylose isomerase (•,▪,▴,⋄) or without xylose isomerase (◯,□,Δ,⋄). Error bars represent the 95% confidence intervals.

While not wishing to be bound to any particular theory, it is believed that xylose isomerase increases cell growth and ethanol productivity for both yeast species Saccharomyces pastorianus and Saccharomyces cerevisiae due to enhanced glucose consumption rates. The mechanism by which xylose isomerase improves cell growth and ethanol productivity appears to be related to the conversion of glucose to fructose by the enzyme. Additionally, these results demonstrate that Saccharomyces pastorianus has the potential to convert cellulosic derived sugars to ethanol at near theoretical conversion levels while obtaining higher biomass concentrations than Saccharomyces cerevisiae. Since one income source for bioethanol production is the cell mass, the higher Saccharomyces pastorianus biomass has the potential to enhance the overall economics of the process. Additionally, the incorporation of immobilized xylose isomerase may also have positive impacts on recombinant Saccharomyces cerevisiae ethanol yields and productivity.

As shown by the FIGS. and Tables 1 and 2, the use of a multiple fermentation process involving both yeast and bacterial fermentation in conjunction with added enzymes may be used to provide improved ethanol production by fermenting sugars that are typically not subject to existing methods of fermentation in order to improve ethanol yields. This may result in various savings including not having to purchase yeast for every fermentation run, faster conversion of glucose to ethanol by, for purposes of example only and not intended to be limiting, combination of xylose isomerase and killed Escherichia coli slurry, as well as the conversion of xylose to ethanol, indirectly, to increase the overall yield of the fermentation process. Further, the process allows one to avoid using recombinant yeast species, which in turn allows the by-products of the fermentation process to be sold as feed for livestock without fear of introducing recombinant organisms into the food chain.

The disclosed subject matter may be better understood with reference to the examples, below.

### Example 1

The process was based on a 1000 liter (L) basis. Hydrolyzed lignocellulosic sugars, comprising 300 kg glucose and 150 kg xylose were combined with 0.025 kg yeast, Saccharomyces pastorianus, obtained from Winemakeri, Inc. (Beaverbank, NS, Canada) and commercially sold as Liquor Quik Super Yeast X-Press and introduced into a fermenter containing 5 kg of xylose isomerase and allowed to ferment for 30 hours. The xylose isomerase was purchased from Sigma and is marketed as glucose isomerase obtained from Streptomyces murinus. The manufacturer is Novozyme Corporation under the trade name Sweetzyme® IT Extra. The immobilized enzyme and supporting material was used at a concentration of 5 g/L. The manufacturer states that the enzyme is active for over 200 days under the glucose isomerization conditions used in the high-fructose corn syrup industry.

After the yeast fermentation, 166 kg of ethanol, 150 kg of xylose and 50 kg of yeast of yeast were present and 150 kg of CO₂ were produced. The mixture underwent solid/liquid separation via centrifugation in order to separate the yeast from the ethanol and xylose mixture. (Other solid/liquid separation methods, such as filtration, may also be used.) The ethanol/xylose mixture then underwent distillation via a standard glass distillation system on a 100 mL scale to separate the ethanol from the xylose. 158 kg of ethanol was obtained from this process.

The remaining 8 kg of ethanol and 150 kg of xylose were then introduced to a bacterial fermentation vessel. This was allowed to undergo bacterial fermentation in the presence of Escherichia coli MG1655 (ATCC 700926) with a starting mass of 0.0375 kg with an initial volume of 500 L for 24 hours. The Escherichia coli were grown at 37° C. in Fed-Batch mode using an exponential feed profile in a BioStatB 5-L vessel from Sartorius, any computer or analog controlled fermenter, as known to those of skill in the art, would be acceptable with pH, T and dissolved oxygen controlled. The exponential growth rate was set up to 0.15 h⁻¹. Dissolved oxygen was set to 30% saturation and pH was set to 6.9 using standard fed-batch control algorithms for control. 84 kg of CO₂ and 8 kg of ethanol were released from this process. The remaining bacterial slurry was then heat killed by raising the temperature of the fermenter vessel to 65° C. for one hour to produce a heat-killed bacterial slurry containing 66 kg of dry weight in 750 to 1500 L of heat-killed bacterial slurry. This heat-killed bacterial slurry was then introduced to a second yeast fermentation process, using the same fermenter vessel, converting glucose to ethanol and xylose provided by cellulosic sugars in the hydrolysates using xylose isomerase. The products from this second yeast fermentation included yeast, ethanol, xylulose, and xylose.

### Example 2

Corn stover hydrolysate was used to examine ethanol productivity using the E. coli cell extracts (ECE) and xylose isomerase (XI) with the yeast Saccharomyces pastorianus in a system as described herein. Corn stover hydrolysates were prepared from finely ground corn stover pretreated by the Alkaline Hydrogen Peroxide (AHP) method and was subsequently enzymatically hydrolyzed to release glucose and xylose. The total volume of corn stover hydrolysate was 230 mL. The experimental design was triplicate cultures of 30 mL. Control cultures using pure sugars (SH) were conducted at this smaller scale and with these lower concentrations of sugars. The sugar concentrations in the pure sugar cultures (synthetic hydrolysate) were matched to the sugar concentrations in the corn stover hydrolysate. E. coli extract was added at 31.5 g dcw/L and the xylose isomerase was added at 5 g/L. To quantify the enhancement in glucose consumption by S. pastorianus due to the ECE, cultures were examined with and without ECE, with and without the xylose isomerase (XI), and with and without minimal medium (MM). Error bars represent 95% confidence intervals.

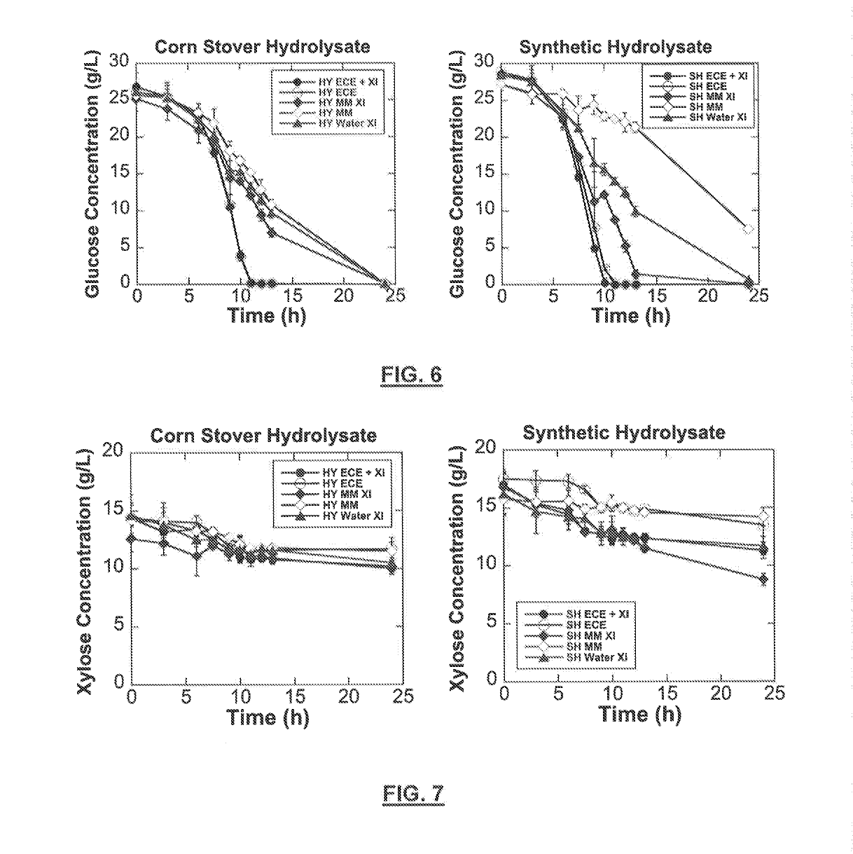

As shown in FIG. 6, S. pastorianus was able to rapidly consume the glucose in the corn stover hydrolysate (HY) and synthetic (pure) biomass sugars (SH) in the presence of E. coli extract (ECE). The addition of xylose isomerase (XI) slightly improved the glucose consumption time for the cultures with synthetic biomass sugars; however had no effect on the corn stover hydrolysate ECE combination. Additionally, the addition of minimal medium was not necessary for the corn stover hydrolysate. Interestingly, the xylose isomerase increased glucose consumption in the pure biomass sugars more so than the minimal medium addition.

FIG. 7 shows the xylose profiles. As expected, the xylose concentration for the pure biomass sugars reached equilibrium at approximately 20%; however, in the corn stover hydrolysate, the xylose isomerase was less effective at converting xylose. More importantly, the ethanol productivity in the corn stover hydrolysate was equal to that of the pure biomass sugars (FIG. 8). Additionally, in the corn stover hydrolysate, the xylose isomerase was not needed to reach these higher ethanol productivity levels, which is will decrease production costs.

### Example 3

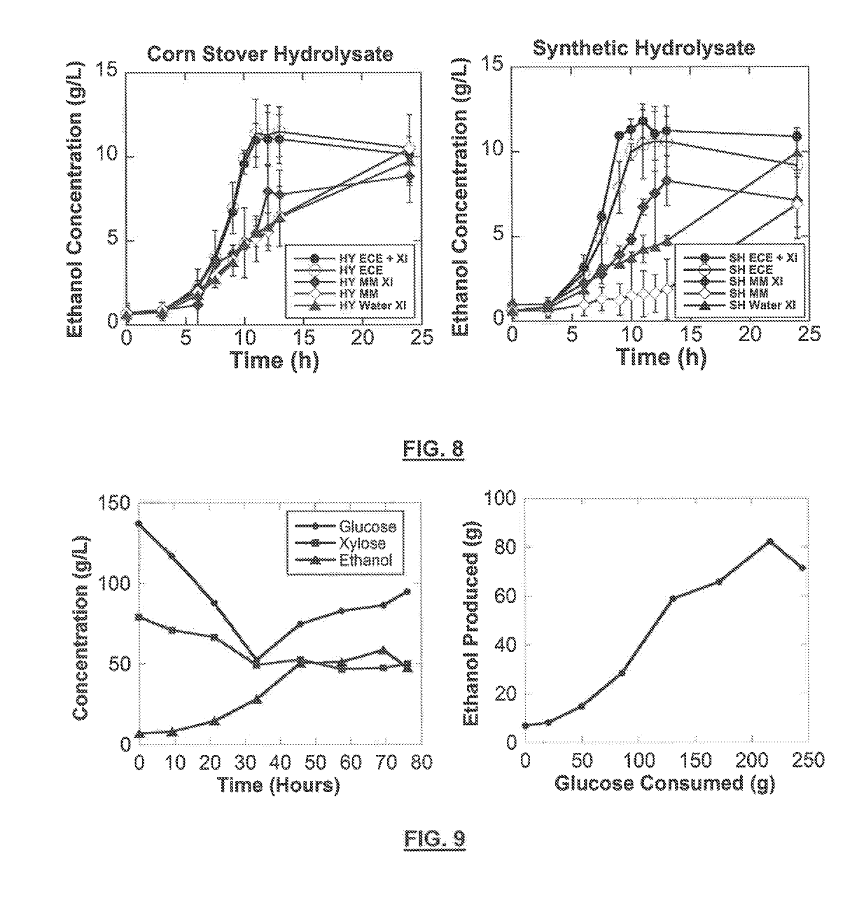

S. pastorianus was cultured in an un-optimized fed-batch fermentation using the heat-killed ECE and synthetic biomass sugars. The fermentation contained minimal media with 150 g/L glucose, 75 g/L xylose, and 31.5 g dcw/L E. coli cell extract (ECE). At 20 hours, 5 g/L xylose isomerase was added to the fermenter vessel. The glucose (500 g/L) feed started at 32 hours and was linear, such that the volume of the liquid increased by 150% from 32 to 76 hours. The glucose feed was slightly higher than the consumption rates, as indicated by the increase glucose levels after 32 hours (FIG. 9). The xylose concentration was maintained at equilibrium concentrations as indicated by the flat curve after 32 hours. The ethanol production levels increased proportionally with the glucose feed until 70 hours when the glucose consumed and total ethanol produced values are calculated (FIG. 9).

While the subject matter has been described in detail with respect to the specific embodiments thereof, it will be appreciated that those skilled in the art, upon attaining an understanding of the foregoing, may readily conceive of alterations to, variations of, and equivalents to these embodiments. Accordingly, the scope of the present disclosure should be assessed as that of any appended claims and any equivalents thereto.

What is claimed is:

1. A multiple stage fermentation process comprising:
contacting a mixture comprising multiple sugars with one or more strains of yeast, a first portion of the sugars being converted by the yeast according to a first fermentation process;
contacting a second portion of the sugars with one or more strains of bacteria, the second portion of the sugars being converted by the bacteria according to a second fermentation process; and
contacting at least one of the first portion of the sugars and the second portion of the sugars with an enzyme, wherein one or more of the sugars is a substrate for the enzyme.

2. The process of claim 1, wherein the multiple stage fermentation process produces an alcohol.

3. The process of claim 2, wherein the alcohol is ethanol.

4. The process of claim 1, wherein the first fermentation process includes an aerobic period and an anaerobic period.

5. The process of claim 1, wherein the second fermentation process includes an aerobic period and an anaerobic period.

6. The process of claim 1, wherein the multiple sugars include sugars having from three to seven carbon atoms in their structure.

7. The process of claim 6, wherein the multiple sugars include glucose and xylose.

8. The process of claim 1, further comprising obtaining the mixture comprising multiple sugars from a feedstock.

9. The process of claim 8, wherein the feedstock is a cellulose biomass.

10. The process of claim 8, wherein the feedstock contains lignocellulose.

11. The process of claim 8, further comprising hydrolyzing the feedstock.

12. The process of claim 1, wherein the one or more strains of yeast is from the genus Saccharomyces or the genus Candida.

13. The process of claim 12, wherein the one or more strains of yeast comprises at least one of Saccharomyces cerevisiae and Saccharomyces pastorianus.

14. The process of claim 1, wherein the enzyme is a xylose isomerase, a hemicellulase, or a cellulase.

15. The process of claim 1, wherein the bacteria comprise bacteria of the genera Escherichia, Salmonella, Bacillus, or Zymomonas.

16. The process of claim 1, further comprising recovering the yeast following the first fermentation process.

17. The process of claim 1, wherein the first portion of sugars is contacted with the one or more strains of yeast prior to contacting the second portion of sugars with the one or more strains of bacteria.

18. The process of claim 1, wherein the first portion of sugars is contacted with the one or more strains of yeast simultaneously with contacting the second portion of sugars with the one or more strains of bacteria.

19. The process of claim 1, further comprising killing the bacteria following the second fermentation process.

20. The process of claim 1, further comprising repeating the process in a recycle fermentation process.
